(19) United States
(12) Patent Application Publication
(10) Pub. No.: US 2016/0325050 A1
(43) Pub. Date: Nov. 10, 2016

(54) DEVICE FOR DISPENSING A SUBSTANCE, IN PARTICULAR A MEDICINE

(71) Applicant: SHARPSIGHT B.V., Herten (NL)

(72) Inventor: Arnaldo GONÇALVES, HERTEN (NL)

(21) Appl. No.: 15/110,196

(22) PCT Filed: Jan. 7, 2015
(86) PCT No.: PCT/NL2015/050007

§ 371 Date: Jul. 7, 2016

(30) Foreign Application Priority Data
Jan. 7, 2014 (NL) 2012054

(51) Int. Cl. A61M 5/315; B05C 17/01

(52) U.S. Cl. CPC A61M 5/31576; B05C 17/012; B05C 17/0146; A61M 2005/3101

(57) ABSTRACT

A device designed for administering a substance, in particular a medicine, to a part of a human or animal body, comprising a housing provided with a reservoir for holding a certain amount of substance. The reservoir includes a dispensing opening, which is closed in the inactive state, for dispensing the substance from the reservoir, as well as dispensing means for dispensing, in use, an amount of substance via the dispensing opening whilst simultaneously releasing the dispensing opening temporarily.

The invention relates to a device designed for administering a substance, in particular a medicine, to a part of a human or animal body, comprising a housing provided with a reservoir for holding a certain amount of substance.

Such dispensing devices are generally known, usually they are made available to persons in the form of tubes provided with a medicinal substance (such as a liquid or a gel or an ointment), which persons are thus responsible themselves for administering the substance (to themselves or to a direct relation such as a partner or a child). Consider in this connection self-medication by administering a particular, read medically desired, amount to an eye, mouth, nose, ear or another part of the body of a human or an animal.

Accordingly, the invention as described in this patent application is not limited solely to use in or on an eye, mouth, nose or ear, but it may also be used in or on other parts of the human and/or animal body.

A drawback of such a dispensing device is that the user often has little or no experience in using such a dispensing device in a correct way. As a result of this lack of expertise, unaided (regular) administration of such a substance, for example to an eye, mouth, nose or ear, using a known dispensing device, invariably leads to incorrect amounts (too little or too much) of the substance being dispensed, which effect is further reinforced in the case of administration to an eye, mouth, nose or ear by the user's unnatural position, which is difficult to control, upon administering the medicine to his own eye.

Another drawback is the fact that contagion and contamination of the substance via the dispensing opening frequently occurs with the known dispensing device, which is not conducive to the healing process of the affected body part (for example an eye, mouth, nose or ear).

The object of the invention is to provide an improved dispensing device, and in order to achieve that object the reservoir comprises a dispensing opening—which is closed in the inactive state—for dispensing the substance from the reservoir, as well as dispensing means for dispensing—in use—an amount of substance via the dispensing opening whilst simultaneously releasing the dispensing opening temporarily.

More particularly, the dispensing opening can be closed by means of a sealing cap in the inactive state, which sealing cap releases the dispensing opening temporarily in use under the influence of the dispensing means. Contagion and contamination of the substance via the dispensing opening is thus prevented, which is conducive to the healing process.

In a functional embodiment, the dispensing opening is provided in the sealing cap, which sealing cap surrounds the reservoir, whilst the reservoir further includes a projecting pin element, which extends into the dispensing opening in the inactive state.

In one embodiment, the dispensing means comprise a displacement element that can extend into the reservoir, wherein the reservoir is cylindrical in shape, for example, and the displacement element is configured as a piston element.

More specifically, the dispensing means comprise an engaging rod which is movably accommodated in the housing, wherein the engaging rod engages the sealing cap on the one hand and the displacement element on the other hand, and wherein the engaging rod engages the displacement element via a toothed transmission. This makes it possible to dispense the substance from the dispensing device in a simple meaner by means of the displacement element whilst simultaneously releasing the dispensing opening.

More in particular, in one embodiment the toothed transmission comprises a rack-and-pinion transmission, wherein the displacement element is provided with a toothing, wherein the engaging rod comprises an engaging cam which engages the teeth of the displacement element and wherein the engaging cam is movable, in particular against a spring force.

More specifically, in one embodiment the engaging rod is provided with several recesses, in which a first gear forming part of the toothed transmission engages, wherein the gear transmission comprises a second gear connected to the first gear by means of a gear shaft, which second gear engages the displacement element.

In a special embodiment, the dispensing means comprise a dispensing pawl that can engage in a slot of the housing and on the engaging rod, which dispensing pawl is movable against a spring force between a starting position and an end position.

In its starting position, the dispensing pawl can be moved into engagement with an engaging cam provided on the engaging rod.

To make repeated dispensing or use of the dispensing device according to the invention possible, the housing is provided with a first cam, which first cam moves the dispensing pawl out of engagement with the engaging cam near its end position, and also with a second cam, which second cam moves the dispensing pawl into engagement with the engaging cam near its starting position.

The sealing cap in that case releases the dispensing opening against a spring force, so that it will close again under the influence of the spring force after use, thereby preventing undesirable contamination of the substance (in the case of a dispensing device that is otherwise open or exposed to the atmosphere).

In one embodiment, the sealing cap further surrounds the dispensing opening, whilst on the other hand a spring element is provided between the sealing cap and the housing. More specifically, the spring element is provided around the reservoir.

The invention will be explained in more detail below with reference to a drawing, in which:

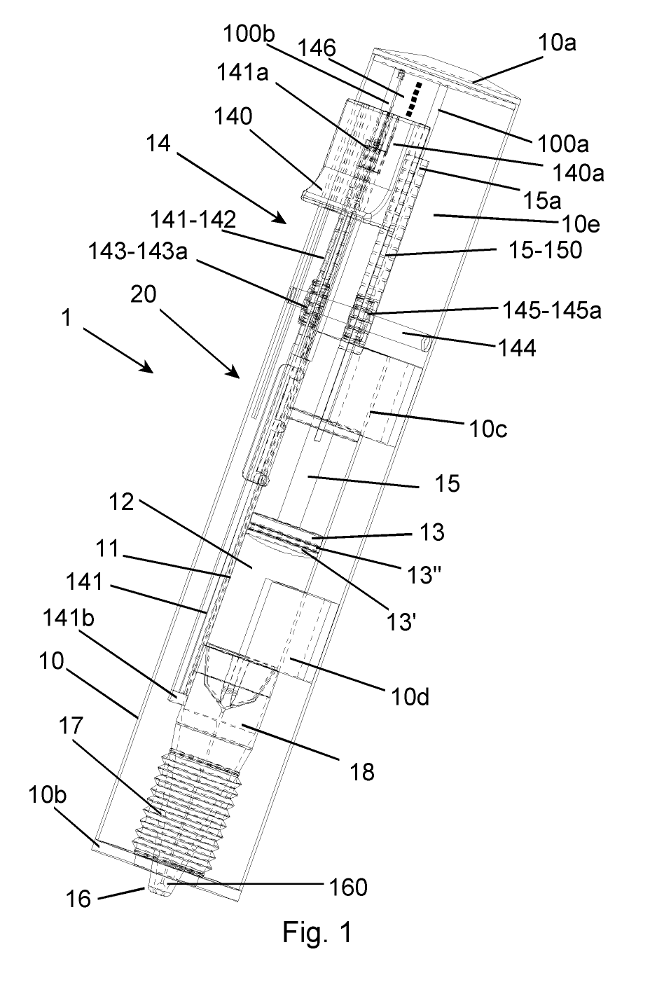

FIGS. 1-6 show various views of an embodiment of a dispensing device according to the invention.

For a better understanding of the invention, corresponding parts will be indicated by identical numerals in the description of the figures below.

FIG. 1 indicates an embodiment of a dispensing device 1 made up of a housing 10 having a first end face 10a and a second end face 10b. The shape or the dimensions of the housing 10 are such that it is easy to hold and handle by a user. The housing 10 includes a reservoir or chamber 11 for holding a certain amount of substance. The substance can be a medicine, for example, to be administered to a part of a human or animal body, such as an eye, mouth, nose or ear, for example. For easy reference, the substance is indicated by numeral 12.

It should be noted that the dispensing device 1 as described in this patent application can either be empty or be prepared already with a certain amount of substance 12, which is contained in the chamber 11 in question. The chamber or reservoir 11 is preferably configured as a cylinder space which houses a displacement element 13. The chamber 11 may also have a rectangular or other cross-sectional shape, however.

The displacement element 13 may in that case be configured as a piston element or plunger 13, which can be moved up and down in the chamber 11 by means of a plunger rod 15. The plunger 13 may thus be provided with a circular recess 13′ at its circumferential edge, in which a sealing ring 13″ can be placed. As a result, the plunger 13 seals accurately against the inner wall of the cylindrical chamber 11, preventing leakage of substance 12 behind the plunger 13.

The plunger rod 15 is accommodated in a supporting part 10c that forms part of the housing 10 and extends in the direction of the first end face 10a. In a functional embodiment, the movable piston rod 15 is hollow and is guided over a guide shaft or support shaft 100a, which extends from the first end face 10a into the open space 10e of the housing 10. The hollow piston rod 15 is thus guided over the support shaft 100a and through the housing part 10c.

Near the second end face 10b, the dispensing device 1 is provided with a dispensing opening 16 which is in communication with the chamber 11 for dispensing a particular amount of substance 12. In the inactive state, the dispensing opening 16 is closed by a sealing cap 160, which can be moved while dispensing by dispensing means 14 (yet to be described), so that the dispensing opening 16 is released for dispensing the substance 12.

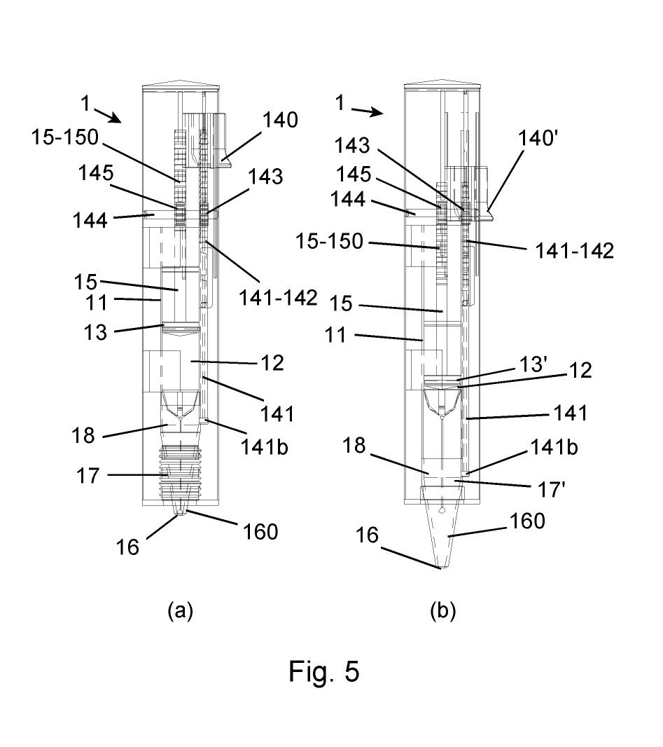

According to the invention, the dispensing device is also provided with dispensing means 14, which are configured to dispense a particular amount of substance 12 via the dispensing opening 16 in use whilst simultaneously opening or releasing the dispensing opening. More specifically, the dispensing means 14 are configured to move the sealing cap 160—which closes the dispensing opening 16 in the inactive state—so that the dispensing opening 16 is released (temporarily) for dispensing a particular amount of substance 12. See also FIGS. 5a and 5b.

The dispensing means 14 are in part made up of a dispensing pawl or sliding knob 140, which is slidably accommodated in a slot (not shown) in the housing 10. The dispensing pawl or sliding knob 140 is also accommodated in a guide 101-102 in the housing 10. The guides 101 and 102 are formed by slots or recesses provided in the inner wall that forms the housing 10.

The dispensing pawl or sliding knob 140 is provided with a stop or engaging cam 140a which, upon movement of the sliding knob 140 (for example by a user's thumb), engages a first end 141a of an engaging rod 141 that extends in the longitudinal direction in the housing 10. The engaging rod 141 extends in the longitudinal direction in the housing 10 and engages, with its other end 141b, a (flexible or non-flexible) part 18 which is connected to the sealing cap 160 via a spring element 17. The engaging rod 141 is provided with a toothing 142 near its first end 141a, which engages or meshes with teeth 143a of a first gear 143.

Said first gear 143 is provided on a shaft 144 which is oriented transversely to the engaging rod 141 and which is rotatably accommodated in the housing 10. A second driving wheel 145 is placed on the same shaft 144, which driving wheel is provided with a toothing 145a which engages or meshes with a corresponding toothing 150 on the piston rod 15.

FIG. 5a shows the situation of the dispensing device in the starting position of the dispensing stroke, just before dispensing takes place. When the sliding knob 140 is moved along with its guide 101-102 by a user's thumb of finger, this will result in the first end 141a of the engaging rod 141 being engaged by the cam 140a. The engaging rod 141 is thus pushed or moved in the direction of the second end face 10b in the housing 10, causing the first gear 143 to be rotated by means of the gear transmission 142-143a.

Accordingly, the simultaneous rotation of the second gear 145 results in the piston rod 15 being engaged and moved over the support shaft 100a and through the housing part 10c and a corresponding movement of the displacement element 13 in the chamber 11. In addition to effecting a movement of the displacement element 13 within the chamber 11, the movement of the engaging rod 141 also effects a simultaneous movement of the sealing cap 160 (through the other end 141b), causing the dispensing opening 16 to be released.

The release of the dispensing opening 16 is made possible in that the connecting rod 141 engages the flexible part 18 and the sealing cap 160 with its other end 141b, which sealing cap will open against a spring force in the dispensing opening 16 as a result of this action. In this embodiment, the spring force is provided by a spring element 17, which is connected as a bellows construction to the sealing cap 160 on the one hand and to the flexible part 18 on the other hand.

In FIG. 5a the spring element 17 is shown in the released condition (with the dispensing device in the starting position of the dispensing stroke), whilst in FIG. 5b, which shows the dispensing device in the end position of the dispensing stroke, the spring element (now indicated at 17′) is compressed and tensioned. In FIG. 5b the sliding knob 140 is furthermore shown in its end position (indicated at 140′), and the piston 13 (indicated at 13′) has likewise been moved within the chamber 11.

The dispensing device according to the invention is in particular very suitable for the repeated administration, in succession or not in succession, of an exact amount of substance from a reservoir through the dispensing opening 16, for example to a human or animal eye, mouth, nose or ear (or another part of the body). Such a precise and repetitive dispensing amount of medicine makes it possible also for less qualified or non-qualified persons to use the dispensing device 1 without the risk of too little or too much of the medicine being administered to the relevant part of the human or animal body.

The specific construction of the dispensing means enables the non-qualified user to use the dispensing device 1 and administer the same predetermined amount of medicinal substance 12 to the relevant part of the human or animal body (i.e., mouth, nose, ear, etc) via the dispensing opening 16 with every dispensing action without having received extensive instructions.

The dispensing means 14 are for that purpose provided with a dose setting mechanism, which is indicated by reference numeral 20 in the figures. Said dose setting mechanism 20 is formed by a stop 200a provided in the housing 10, which stop 200a makes contact with a corresponding stop 140b that forms part of the dispensing pawl or sliding knob 140. The contact between the stop 200a and the stop 140b takes place at the moment when the sliding knob 140 is moved along the guide 101-102 in the direction of the stop 200a, against the spring force of the spring element 146, by the user. The spring element 146 is connected to the dispensing ball/sliding knob 140/cam 140a on the one hand and to the first end face 10a on the other hand.

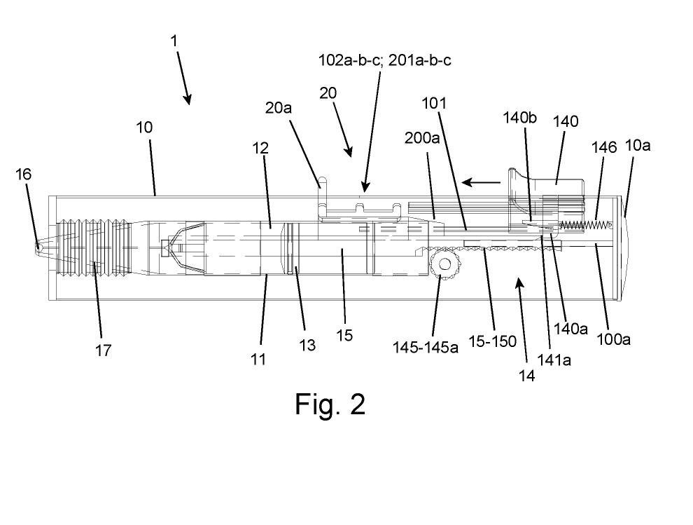

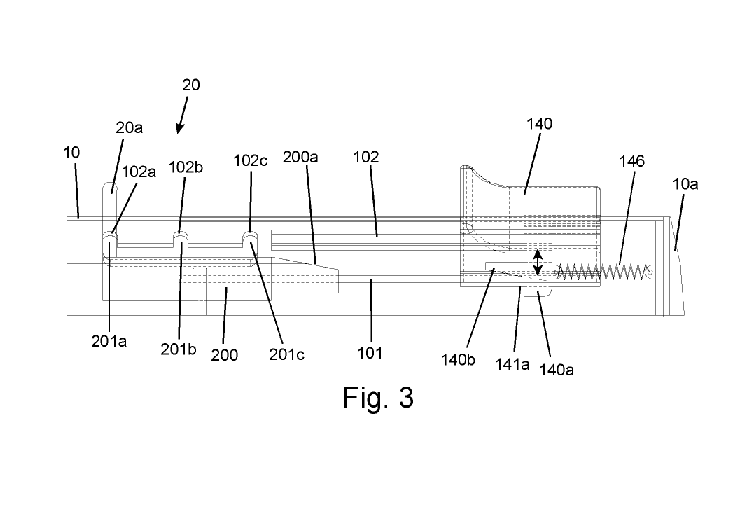

Because of the bevelled shape of the stop 200a, the sliding knob 140—which is in the end position of the movement effected by the user (see reference numeral 140′ in FIG. 5b)—is moved upward out of the guide 101-102, after which the engaging rod 141 moves back to its starting position under the influence of the tensioned spring element 17 (which tensioned spring element is indicated by reference numeral 17′ in FIG. 5b) and the dispensing pawl or sliding knob 140 freely moves back to the starting position shown in FIGS. 2 and 3 (and 5a) under the influence of the tensioned spring element 146. Once it has been returned to the starting position shown in FIGS. 1-2-3-5a, the dispensing pawl or sliding knob 140 will move back into the guides 101-102 again.

As a result, the dispensing pawl or sliding knob 140 can be moved over a (constructionally) predetermined distance, against the spring force of the spring element 146, by the user from its starting position (shown in FIGS. 2 and 3 and 5a), via the guide 101-102, in the direction of the stop 200a (being the end position 140′ in FIG. 5b). The cam 140a will again carry the engaging rod 140 along, so that the displacement element 13 is moved in the chamber 11 via the gear transmission 143-144-145 whilst simultaneously the dispensing opening 16 is released. In this way it is possible to dispense the same amount of substance 12 several times in succession in an efficient manner, with the displacement element being moved over the same distance within the chamber 11 each time an amount of substance is dispensed.

In another embodiment it is possible to set the dispensing movement or dispensing stroke of the dispensing pawl or sliding knob 140 because the dose setting mechanism 20, and in particular the stop 200a, can be moved over some distance with respect to the starting position of the sliding knob 140. The dose setting element 200 and the stop 200a are for that purpose provided with one or more setting cams 201a-201b-201c, which can be received in corresponding recesses 102a-102b-102c provided in the housing 10.

In this embodiment, the dose setting mechanism 20 can be set to three discrete dispensing positions, using the setting knob 200a, by moving the dose setting element 200 within the housing 10, such that the setting cams 201a-201b-201c can be received in one of the recesses 102a-102b-102c. The greatest distance over which the sliding knob 140 can be moved (and accordingly the largest dispensing stroke of the displacement element 13 and consequently the largest amount of substance to be dispensed) can be realised by receiving the setting cam 201c in the recess 102a. It will be apparent that embodiments in which the setting knob 200a can be set to two, three or more discrete dispensing positions, depending on the dispensing application, are also conceivable.

In this setting, the sliding knob 140 must travel the longest distance between its starting position and its own position along the guide 101-102 before—in the end position, the stop 200 moves the sliding knob 140 upward and out of the guide 101-102 by means of the stop 140b, after which the engaging rod 141 moves back to its starting position under the influence of the spring element 17 and the dispensing pawl/sliding knob 140 moves back to its starting position in the direction of the first end face 10a under the influence of the spring force of the spring element 146. At the location of the first end face 10a, the housing is provided with a further camera (not shown), which moves the sliding knob 140 back into the guide 101-102.

During the return movement of the dispensing pawl or sliding knob 140, the cam 140a no longer bears against the first end 141a of the engaging rod 141, so that also the drive shaft will move back in the direction of the end face 10a in the housing 10 under the influence of the spring force of the spring element 17. As a result, the sealing cap 160 will close the dispensing opening 16 again under the influence of the spring force of the spring element 17, so that contagion/contamination of the substance in the chamber 11 is prevented.

At least the gear 143 is mounted on the shaft 144 via a slip coupling, so that the displacement element 13 will be maintained in its position in the chamber 11 during the return movement of the engaging rod 141 in the housing 10 and will not move back along therewith. The displacement element 13 will therefore maintain the position it takes up in the chamber 11 when the sliding knob 140 moves back from its end position to its starting position, and will consequently be moved one dispensing stroke in the chamber 11 with each subsequent dispensing movement of the dispensing pawl or sliding knob 140, until all the substance has been dispensed from the chamber 11.

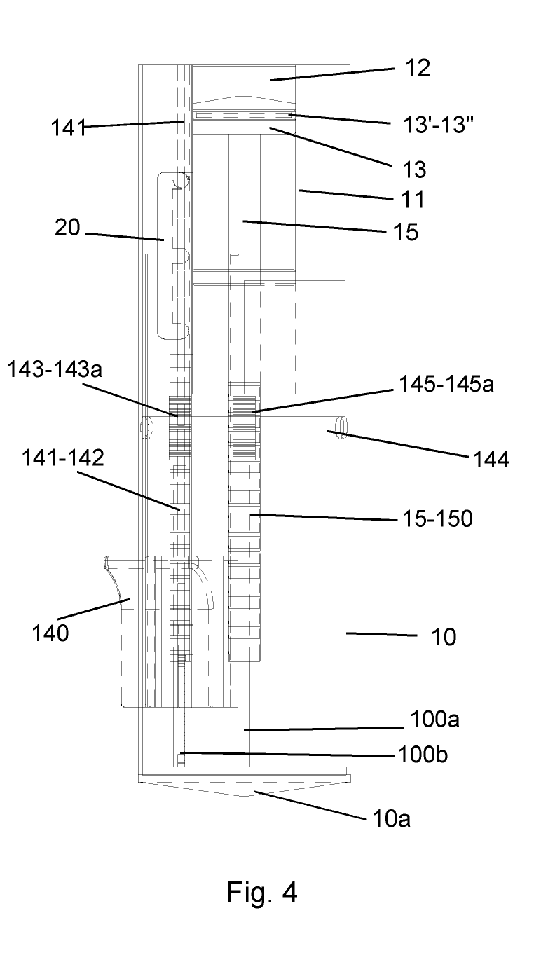

Like the dispensing pawl or sliding knob 140, the engaging rod 141 will come to bear against the cam 140a of the sliding knob 140 upon its return movement, which sliding knob is in its starting position again at that point. Similarly to the construction of the hollow piston rod 15, which is guided over a guide shaft 100a, also the engaging rod may be hollow and be arranged over a support or guide shaft 100b with its first end 141a, which—similarly to the guide shaft 100a—also extends from the first end face 10a into the open space 10e of the housing 10 (see FIGS. 1 and 4). In this way a smooth return movement of the engaging rod 141 to the starting position is ensured, so that the dispensing device will be ready again for a next dispensing action.

It will be understood that, using this construction, the administration of an amount of medical substance from the dispensing opening to the relevant part of the human or animal body is realised in a simple but above all precise manner, which administration can be carried out in a precise, repetitive manner by less qualified persons.

The risk of an incorrect (too little or too much) amount of the substance being dispensed is thus prevented as long as the user moves the dispensing pawl or sliding knob 140 over the entire distance from its starting position to its end position, so that the displacement element 13 is moved over the entire dispensing stroke for dispensing the desired predetermined amount of substance.

The dispensing device according to the invention makes it possible to dispense the medicine to be used in a correct amount and thus use the medicine in question in a more efficient manner, which, in addition, has a cost-saving effect in view of the fact that many medications are relatively costly.

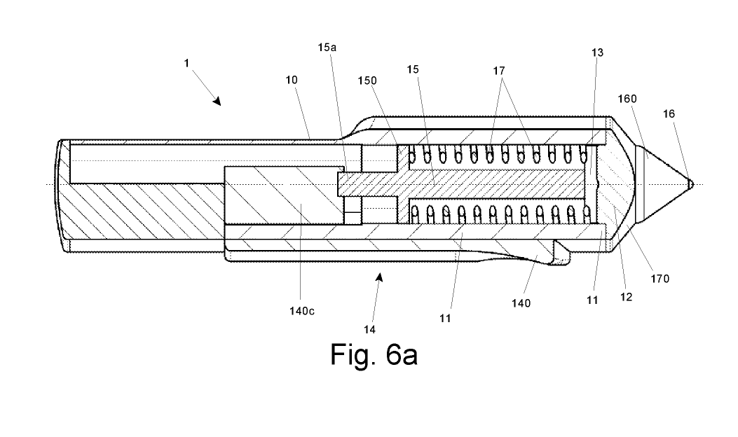

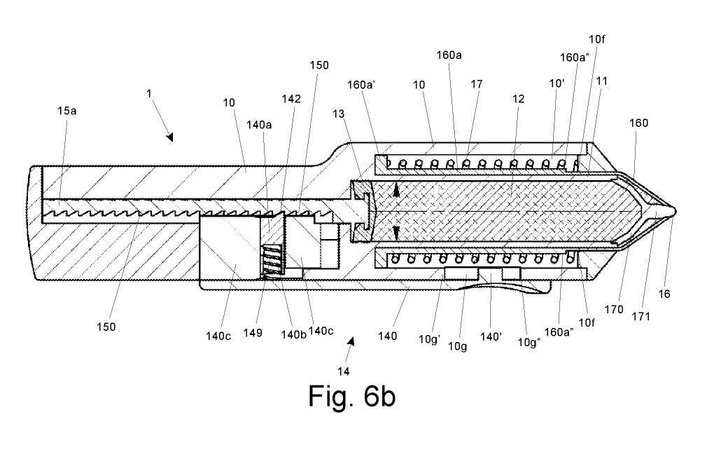

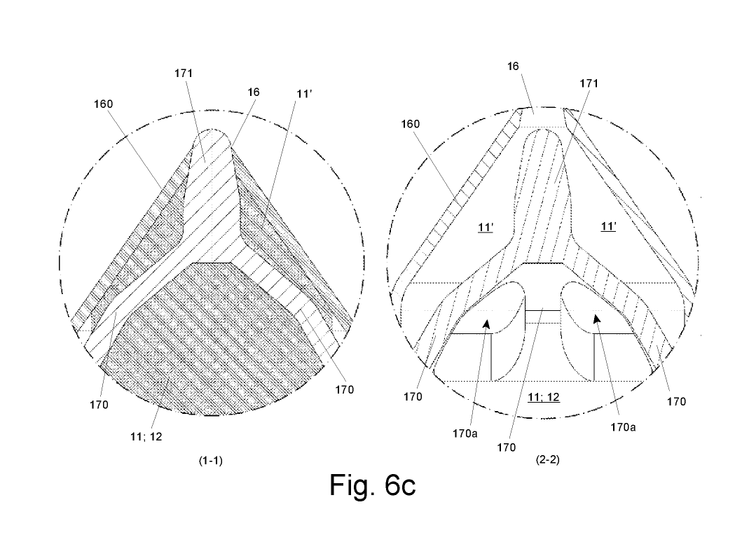

FIGS. 6a-6b-6c show another embodiment of a dispensing device according to the invention, which also makes it possible to dispense the medicine to be used in a correct amount and thus use it in an efficient manner. It is noted that the reference numerals correspond to parts shown in FIGS. 1-5, which parts have a more or less corresponding functionality.

In FIGS. 6a-6b-6c, numeral 1 again indicates the dispensing device, which is made up of a housing 10 including a reservoir or chamber 11 for holding a certain amount of substance. Said substance is indicated at 12.

The chamber or reservoir 11 may be configured as a cylinder space in which a displacement element 13 is accommodated. The displacement element 13 is configured as a plunger 13, which can be moved within the chamber 11 by means of a plunger rod 15. In a similar manner as shown in the embodiment of FIG. 4, for example, the plunger 13 may be provided with a circular recess 13′ at its circumferential edge, in which recess a sealing ring 13″ can be received. This, too, results in an adequate seal against the inner wall of the cylindrical chamber 11, thereby preventing leakage of substance 12 behind the plunger 13. As is clearly shown in FIG. 6b, the plunger rod 15 is provided with a toothing 150 and extends into the housing 10 with its plunger rod end 15a.

The dispensing means 14 comprise a dispensing pawl or sliding knob 140 which is slidably accommodated in the housing 10. The dispensing pawl or sliding knob 140 is provided with a carrier block 104c provided with a recess, in which a stop or engaging cam 140a is accommodated. Upon movement of the sliding knob 140 in the direction of the dispensing opening 16, the engaging cam 140a is carried along by the carrier 140c.

The engaging cam 140a is provided with a toothing (a tooth) 142, which engages the toothing 150 of the piston rod 15. The transmission mechanism between the dispensing pawl 140 and the piston rod 15 can be defined as a rack-and-pinion transmission, since movement of the dispensing pawl 140 in the direction of the dispensing opening 16 will result in translational movement (effected by the carrier 140c) of the engaging cam 140a and, as a result of the fact that the toothing 142 engages in the toothing 150, also in the piston rod 15 and the displacement element 13 being carried along in the cylindrical space or chamber 11.

The dispensing pawl 140 is provided with a stop 140′ which is accommodated in a slot 10g in the housing 10. The slot 10g is provided with a first, rear stop 10g′ and a second, front stop 10g″. In particular the second, front stop 10g″ serves to limit the forward movement of the dispensing pawl 140 (and in particular the stop 140′) and accordingly also the forward movement of the piston rod 15 and the displacement element 13 in the space 11 (and accordingly the maximum amount of substance 12 to be displaced when dispensing takes place) in the direction of the dispensing opening 16.

It should be noted that the amount of substance 12 that can be delivered during a dispensing action is determined on the one hand by the maximum movement of the dispensing pawl 140 in the recess 10g (the distance the stop 140′ can travel in the recess 10′ before being stopped by the front stop edge 10g″) or, on the other hand, by the dimension of the toothing of the toothed transmission shown in FIGS. 1-5 or the dimension of the teeth of the rack-and-pinion transmission in the embodiment shown in FIGS. 6a-6b-6c.

More specifically, in the embodiments shown in FIGS. 6a-6b-6c the dispensing opening 16 is specifically configured as being an opening in a sealing cap 160 that can be moved in the longitudinal direction of the dispensing device 1 against the spring force of a spring 17. By this is meant that the sealing cap 160 is movable in the same direction as the displacement element 13 within the cylindrical space 11. The sealing cap 160 is for that purpose extended with a sleeve-like end 160a, which is provided around the cylindrical space 11. The sleeve-like end 160a of the sealing cap 160 is provided with a stop boss 160a′, against which a spring element 17 abuts.

The spring element 17, which comprises several windings around the cylindrical chamber 11 and the sleeve-like end 160a, is accommodated in a cylindrical interspace 10′ between the housing 10 and the cylindrical chamber 11. The spring element 17 is accommodated (or clamped) between the circumferential stop boss 160a′ of the sleeve-like end 160a and an internal stop 10f of the interspace 10′.

As is clearly shown in FIG. 6b, the sleeve-like end 160a of the sealing cap 160 is also provided with a circumferential stop boss 160a″, which limits movement in the longitudinal direction of the sealing cap 160 (against the spring force of the spring element 17) in that the forward movement of the sealing cap 160 is stopped by the stop boss 160a″ and the internal stop 10f of the housing 10. This means that the forward movement of the sealing cap 160 is stopped at the moment when the front stop boss 160a″ of the sleeve-like end 160a comes to bear against the stop 10f of the housing 10. As a result, the opening 16 is released, as is clearly shown in FIG. 6c (partial view 2-2).

In said partial view 2-2 of FIG. 6c, the cylindrical space 11 is provided with a projecting pin 171 near the dispensing opening 16, which pin is connected to the circumferential wall of the space 11 by means of support members 170. In a closed position as shown in FIG. 6b and the partial view 1-1 of FIG. 6c, the projecting pin 171 closes the dispensing opening 16. In order to make dispensing the substance 12 possible, the sealing cap 160 moves against the spring force of the spring element 17, as a result of which the sealing cap 160 will move clear of the projecting pin 171 and the dispenser opening 16 will thus be released for dispensing a particular amount of substance 12 that has collected in the subspace 11′ of the space 11. The support members 170 are for that purpose provided with openings 170a which make is possible for substance 12 to be moved or displaced from the space 11 to the subspace 11′ and, accordingly, from the released opening 16.

The dispensing principle of this embodiment as shown in FIGS. 6a-6b-6c is as follows. Upon actuation of the dispensing pawl 140, the sealing cap 160 moves against the spring force of the spring element 17, causing the sealing cap 160 to move clear of the projecting pin 171. Simultaneously therewith, the piston rod 15 and the displacement element 13 will be moved by the rack-and-pinion transmission (engaging cam 140a engaging the toothing 150), so that part of the substance 12 is displaced to the space 11′ of the space 11, which subspace 11′ is created by the forward movement of the sealing cap 160.

At the moment when the dispensing pawl 140 is released, the spring action of the spring element 17 will cause the sealing cap 160 to move back, as a result of which the amount of substance 12 present in the subspace 11′ is dispensed through the dispensing opening 16. The sealing cap 160 presses the amount of substance 12 from the subspace 11′ through the dispensing opening 16, therefore, until the dispensing opening 16 is closed by the sealing cap 160 as a result of the sealing cap 160 moving over the pin 171.

The substance 12 is thus not dispensed at the moment when the dispensing pawl 140 is actuated by the user (the user's hand), but at the moment when the dispensing means 14 (in particular the dispensing pawl 140) are released by the user. This enables the user to position the dispensing device 1 better (for example with respect to the eye, if the substance to be dispensed is an eye ointment) without other forces influencing said positioning. Having correctly positioned the dispensing device 1, the user can release the dispensing pawl 140, whereupon dispensing of the substance will directly take place.

Likewise, the presence of the rack-and-pinion transmission shown in FIGS. 6a and 6b will result in the carrier element 140c carrying along the engaging cam 140 when the dispensing pawl 140 makes its return movement. The engaging cam 140a is provided with a recess 140b, in which a spring element 149 is accommodated. The engaging cam 140b is thus movably accommodated (or retained) in the space formed in the carrier element 140c (and the dispensing pawl 140) and the toothing 150 of the piston rod 15.

After the dispensing means have been actuated (as a result of which an amount of substance 12 has been moved into the subspace 11′) and the dispensing opening 16 has been temporarily released, said amount of substance 12 will not be dispensed via the released dispensing opening until the dispensing means 14 have been deactivated.

When the dispensing pawl 140 and the engaging cam 140a are moved back again (by the carrier element 140c, which is a fixed part of the dispensing pawl), the engaging cam 140a can move over the teeth 150, with the spring element 149 being compressed each time a tooth is passed. As a result of the counteracting spring force of the spring element 149, however, the engagement between the engaging cam 140a (in particular the toothing 142) and the toothing 150 of the piston rod 15 is maintained so as to make a next dispensing action possible.

1-20. (canceled)

21. A device designed for administering a substance, in particular a medicine, to a part of a human or animal body, comprising a housing provided with a reservoir for holding a certain amount of substance, which reservoir comprises a dispensing opening, which is closed in the inactive state, for dispensing the substance from the reservoir, as well as dispensing means for dispensing, in use, an amount of substance via the dispensing opening whilst simultaneously releasing the dispensing opening temporarily.

22. A device according to claim 21, wherein the dispensing opening can be closed by means of a sealing cap in the inactive state, which sealing cap releases the dispensing opening temporarily in use under the influence of the dispensing means.

23. A device according to claim 22, wherein the dispensing opening is provided in the sealing cap, which sealing cap surrounds the reservoir.

24. A device according to claim 23, wherein the reservoir includes a projecting pin element, which extends into the dispensing opening in the inactive state.

25. A device according to claim 21, wherein the dispensing means comprise a displacement element that can extend into the reservoir.

26. A device according to claim 25, wherein the reservoir is cylindrical in shape and the displacement element is configured as a piston element.

27. A device according to claim 25, wherein the dispensing means comprise an engaging rod which is movably accommodated in the housing, which engaging rod engages the sealing cap on the one hand and the displacement element on the other hand.

28. A device according to claim 27, wherein the engaging rod engages the displacement element via a toothed transmission.

29. A device according to claim 28, wherein the toothed transmission is a rack-and-pinion transmission, wherein the displacement element is provided with a toothing.

30. A device according to claim 29, wherein the engaging rod comprises an engaging cam which engages the teeth of the displacement element.

31. A device according to claim 29, wherein the engaging cam is movable, in particular against a spring force.

32. A device according to claim 28, wherein the engaging rod is provided with several recesses, in which a first gear forming part of the toothed transmission engages.

33. A device according to claim 32, wherein the gear transmission comprises a second gear connected to the first gear by means of a gear shaft, which second gear engages the displacement element.

34. A device according to claim 27, wherein the dispensing means comprise a dispensing pawl that can engage in a slot of the housing and on the engaging rod, which dispensing pawl is movable against a spring force between a starting position and an end position.

35. A device according to claim 34, wherein the dispensing pawl can be moved into engagement with the engaging rod in its starting position.

36. A device according to claim 35, wherein the housing is provided with a first cam, which first cam moves the dispensing pawl out of engagement with the engaging rod near its end position.

37. A device according to claim 35, wherein the housing is provided with a second cam, which second cam moves the dispensing pawl into engagement with the engaging rod near its starting position.

38. A device according to claim 22, wherein the sealing cap releases the dispensing opening against a spring force.

39. A device according to claim 38, wherein a spring element is provided between the sealing cap and the housing.

40. A device according to claim 39, wherein the spring element is provided around the reservoir.
